(19) United States
(12) Patent Application Publication
(10) Pub. No.: US 2015/0185101 A1
(43) Pub. Date: Jul. 2, 2015

(54) DEVICE FOR MEASURING THE PRESSURE OF A GAS IN A POLLUTION CONTROL OR ENERGY STORAGE SYSTEM

(71) Applicant: Inergy Automotive Systems Research (Societe Anonyme), Bruxelles (BE)

(72) Inventors: Jules-Joseph Van Schaftingen, Wavre (BE); Francois Dougnier, Boortmeerbeek (BE); Dominique Madoux, Rumes (BE); Jean-Claude Habumuremyi, Haaltert (BE)

(73) Assignee: Inergy Automotive Systems Research (Societe Anonyme), Bruxelles (BE)

(21) Appl. No.: 14/411,658

(22) PCT Filed: Jun. 28, 2013
(86) PCT No.: PCT/FR2013/051520

§ 371 Date: Dec. 29, 2014

(30) Foreign Application Priority Data
Jun. 29, 2012 (FR) 1256291

(51) Int. Cl. G01L 11/00; G01N 33/00

(52) U.S. Cl. CPC G01L 11/002; G01N 33/0036

(57) ABSTRACT

A device for measuring a pressure of a gas in a pollution control or energy storage system, including: a housing including an inlet and an outlet; a compound disposed inside the housing and configured to absorb at least one portion of gas entering the housing through the inlet, the non-absorbed portion of gas exiting the housing via the outlet; at least one sensor configured to measure a temperature of the compound; a processing unit configured to determine the pressure of the gas by using the temperature measurement from the sensor and a predefined pressure/temperature ratio.

### FIELD OF THE INVENTION

The present invention relates to a device for measuring the pressure of a gas.

The invention applies in particular, but not exclusively, to measuring the pressure inside an energy storage system such as, for example, a hydrogen storage reservoir mounted on a motor vehicle.

The invention applies also, but not exclusively, to measuring the pressure of a gas circulating in a pollution control system intended to reduce the amount of nitrogen oxides (NOx) in the exhaust gases of a motor vehicle. In the remainder of this document, every effort will be made to describe, by way of illustrative example, this particular type of application.

Of course, the present invention applies to measuring the pressure of any type of gas that may be present in a pollution control or energy storage system.

### TECHNOLOGICAL BACKGROUND

The nitrogen oxides present in the exhaust gases of vehicles, in particular diesel vehicles, can be eliminated by a pollution control system using a technique of selective catalytic reduction (generally referred to as SCR). According to this technique, doses of ammonia (NH₃) are injected into the exhaust line upstream of a catalyst on which the reduction reactions take place. Currently, the ammonia is produced by the thermal decomposition of a precursor, generally an aqueous solution of urea. On-board systems for storing, dispensing and metering out a solution of standardised urea (such as that sold under the name Adblue®, a eutectic solution containing 32.5% urea in water) have thus been put on the market.

Another technique consists in storing the ammonia by sorption on a salt, usually an alkaline-earth metal chloride. Generally in this case, the storage system comprises a reservoir designed to contain the salt and a heating device configured in order to heat the salt. Thus, by heating the salt the ammonia is released. A pressure of ammonia is therefore generated. In such an ammonia storage system it is sought to obtain the pressure of ammonia released in order, for example, to verify that it corresponds to a required pressure of ammonia and, where appropriate, carry out corrective actions (for example, by acting on the heating power of the heating device). Generally, a pressure sensor or a pressure regulator is used to measure the pressure of ammonia released. However, these pressure sensors and regulators are expensive and bulky.

### OBJECTIVES OF THE INVENTION

It is therefore desirable to provide a device that makes it possible to measure the pressure of a gas, without using a pressure sensor or pressure regulator.

It is also desirable to provide such a device which is simple to use in a pollution control or energy storage system for a motor vehicle.

It is also desirable to provide such a device which is compact.

It is moreover desirable to provide such a device which is particularly well suited to measuring the pressure of any type of gas that may be present in a pollution control or energy storage system, and in particular ammonia and hydrogen.

### SUMMARY OF THE INVENTION

In one particular embodiment of the invention, a device is proposed for measuring the pressure of a gas in a pollution control or energy storage system comprising:
a casing provided with an inlet and an outlet;
a compound placed inside the casing and being capable of absorbing at least one portion of the gas entering the casing via said inlet, the portion of the gas not absorbed exiting the casing through said outlet;
at least one sensor configured to measure the temperature of the compound;
a processing unit configured to determine the pressure of the gas by using the temperature measurement of the sensor and a predetermined pressure/temperature relationship.

The measuring device according to invention is based on the use of a compound that has an exothermic reaction. The compound is capable of absorbing gas and consequently of generating heat. The gas may be of any type, preferably ammonia or hydrogen.

It is thus proposed to measure the temperature of the compound by means of a temperature sensor or a heat flux sensor, and to deduce therefrom the pressure of the gas on the basis of a pressure/temperature relationship that governs the sorption of the gas on the compound.

The measuring device according to the invention is particularly intended for energy storage or pollution control (for example SCR) systems for motor vehicles. Advantageously, it is possible to use a processor already present on-board the vehicle to act as (i.e. carry out the functions of) the processing unit according to the invention. For example, it is possible to use the processor of the vehicle's on-board computer (sometimes referred to as ECU or engine control unit) or the processor of the control unit of the pollution control system or energy storage system (sometimes referred to as FSCU or fuel system control unit). In this way, the cost of the measuring device according to invention is reduced. Furthermore, by using a processing unit external to the casing, a casing is obtained that is small and easy to assemble. With such a configuration, the measuring device according to invention is less expensive and less bulky than a conventional pressure sensor.

In certain cases, the processing unit may be housed in the casing.

The casing may be made from one or more parts assembled for example by welding. The shape and the dimensions of the casing are generally defined so that the connection of the casing to a component of the pollution control system or energy storage system (duct, reservoir, etc.) requires no or little modification at the component itself. However, an intermediate constituent (or connection end piece) may be placed between the casing inlet/outlet and the component of the pollution control system or energy storage system.

The measuring device according to invention is in particular well suited to the case where the compound (placed inside the casing) is solid. It may be an alkali, alkaline-earth or transition metal chloride. It may be in the pulverulent state or in the form of agglomerates. This compound is preferably an alkaline-earth metal chloride, and very particularly preferably an Mg, Ba or Sr chloride.

Advantageously, the pressure/temperature relationship is a Clausius-Clapeyron relationship.

The Clausius-Clapeyron relationship used by the processing unit may be a theoretical relationship (curve, table, formula, etc.), derived from the literature, preferably validated experimentally. Alternatively, this relationship may be generated experimentally on models and/or prototypes. Such a Clausius-Clapeyron relationship has the advantage of being simple, which results, at the processing unit, in relatively short computing times.

The casing is preferably made of a thermoplastic. Thermoplastics give good results within the context of the invention. The term “thermoplastic” denotes any thermoplastic polymer, including thermoplastic elastomers, and also blends thereof. The term “polymer” denotes homopolymers and copolymers (binary or ternary copolymers in particular). Examples of such copolymers are, non-limitingly: random copolymers, sequential copolymers, block copolymers and graft copolymers. Use may be made of polyamides or polyphthalamides and copolymers thereof, which are preferred for their heat resistance. A blend of polymers or of copolymers may also be used, as can a blend of polymeric materials with inorganic, organic and/or natural fillers such as, for example, but nonlimitingly: carbon, salts and other inorganic derivatives, natural fibers, glass fibers and polymeric fibers. It is also possible to use multilayer structures consisting of firmly attached stacked layers comprising at least one of the aforementioned polymers or copolymers.

Advantageously, the casing comprises an electrical connector via which the sensor is powered and/or via which the processing unit obtains the temperature measurement from the sensor.

Advantageously, the casing comprises means for guiding the gas toward the inlet.

The shape and the dimensions of these guide means are generally defined so that all or some of the gas is directed toward the inlet of the casing. The guide means are, for example, a plate, a tube or a cone. They may be made, for example, of metallic or plastic material.

Advantageously, the casing comprises thermal insulation means.

By using such thermal insulation means, the temperature measurement carried out by the sensor is more accurate.

In one advantageous embodiment, the casing comprises at least one phase-change material (PCM). This makes it possible to limit the potential disturbances of the temperature measurement signal, in particular in the vicinity of the phase-change temperature, and to have a signature, making it possible to ensure that the pressure is above a given value (use of a PCM material) or that the pressure is within a given range (use of 2 PCM materials).

In one particular embodiment, the measuring device according to the invention may be connected to a distribution duct that connects a gas storage reservoir to a dosing module. Preferably, the gas storage reservoir is configured in order to contain a salt on which the gas is stored via sorption, preferably via chemisorption. In this particular embodiment is described in detail below with reference to FIGS. 1 and 2.

In another embodiment, the invention relates to a pollution control system comprising one or more pressure measuring devices as described above.

In another embodiment, the invention relates to an energy storage system comprising one or more pressure measuring devices as described above.

### LIST OF FIGURES

Other features and advantages of the invention will appear on reading the following description, given by way of indicative and nonlimiting example, and the appended drawings, in which:

FIG. 1 illustrates the structural architecture of an SCR pollution control system comprising a gas storage system and a pressure measuring device according to one particular embodiment of the invention;

FIG. 2 illustrates a cross section of the pressure measuring device from FIG. 1.

### DETAILED DESCRIPTION

In the remainder of the description, and by way of example, the gas for which it is desired to measure the pressure is a gas intended to be injected into the exhaust line of a vehicle in order to reduce the amount of nitrogen oxides (NOx) in the exhaust gases. By way of example, the gas is considered to be ammonia. Of course, in an embodiment variant, the gas may be of any other type, and in particular hydrogen.

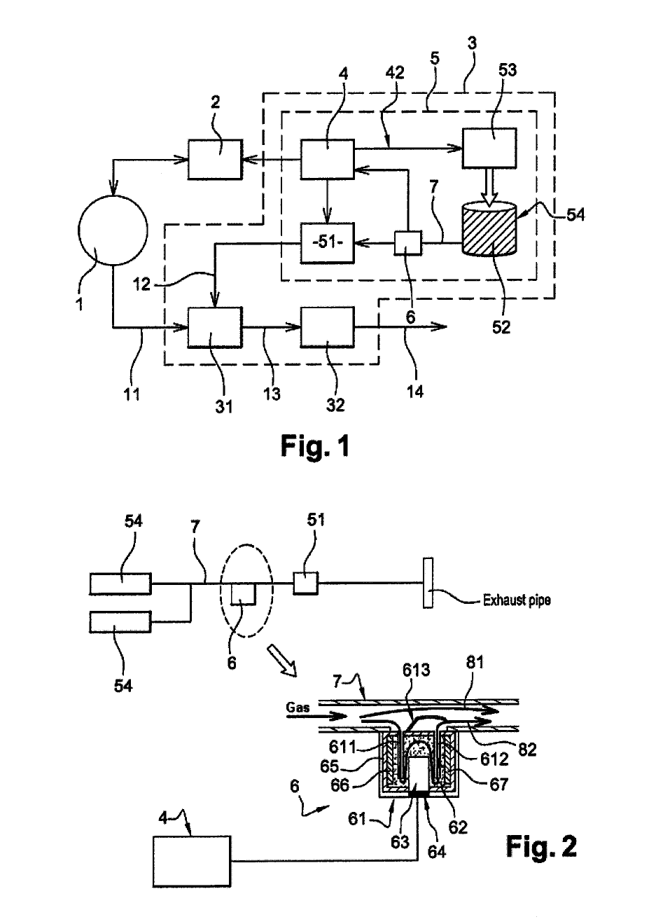

As illustrated in the example of FIG. 1, the engine 1 of the vehicle is controlled by an electronic control unit 2 (sometimes referred to as ECU or engine control unit). The engine 1 cooperates with an SCR pollution control system 3. On leaving the engine, the exhaust gases 11 are directed toward an ammonia injection module 31, in which the ammonia 12 is mixed with the exhaust gases 11. The ammonia/exhaust gases mixture 13 then passes over an SCR catalyst 32 which enables the reduction of the nitrogen oxides (NOx) by the ammonia. The decontaminated exhaust gases 14 are then directed toward the exhaust outlet.

In this exemplary embodiment, the SCR system 3 comprises an ammonia storage system 5. The storage system 5 comprises a reservoir 54, stored in which is a compound 52, for example a solid (and preferably a salt). The ammonia is stored by sorption on the solid 52. The storage system 5 also comprises a control device 4 in charge of controlling a heating device 53 (also referred to as heater) for heating the solid 52 so as to release the ammonia. The heating device 53 may be in the form of an electrical resistor. The ammonia thus released circulates from the reservoir 54 to a dosing module 51, via a distribution duct 7. The dosing module 51 is controlled by the control device 4. In the exemplary embodiment illustrated in FIG. 1, the control device 4 is different from the engine control unit 2. In one embodiment variant, the control device 4 may be integrated into the electronic control unit 2. In another embodiment variant, the control device 4 may be integrated into the system control unit (sometimes referred to as FSCU or fuel system control unit). As illustrated in FIG. 1, a pressure measuring device device 6 according to the invention is connected to the distribution duct 7.

In the embodiment illustrated in FIG. 2, the measuring device 6 comprises a casing 61 provided with an inlet 611 and an outlet 612. Each one of the inlet 611 and outlet 612 communicates with the inside of the distribution duct 7. In this example, the casing 61 comprises a fin (or plate) 613 that extends inside the distribution duct 7. The role of the fin 612 is to guide all or some of the ammonia generated at the outlet of the reservoir 54 toward the inlet 611. In FIG. 2, the fin 613 is configured so that it guides a portion of the gas toward the inlet 611 while leaving the other portion of the gas circulating in the distribution duct 7 following the circulation path indicated by the arrow 81. The portion of ammonia entering the casing 61 passes through, according to the direction of circulation indicated by the arrow 82, a compound 62 placed inside the casing 61. The compound 62 is a solid salt. The compound 62 is capable of absorbing the ammonia entering the casing 61 and of consequently generating heat and of reaching a temperature which is a function of the ammonia pressure. The measuring device 6 also comprises a sensor 63 responsible for measuring the temperature of the compound 62. The sensor may be a temperature sensor or a heat flux sensor. Generally, such sensors are less expensive than a pressure sensor or a pressure regulator or a pressure switch. In one exemplary embodiment, the sensor 63 is a thermocouple. The sensor 63 may be protected in a casing made, for example, from a material that is not very thermally conductive, such as a plastic. The casing 61 is equipped with an electrical connector 64 which is used, inter alia, to power the sensor 63. According to the invention, a processing unit is responsible for estimating the pressure of the stream of ammonia leaving the reservoir 54, on the basis of the temperature measurement from the sensor 63 and a predetermined pressure/temperature relationship. As illustrated in FIGS. 1 and 2, the control device 4 is used to act as the processing unit. Thus, the control device 4 is configured in order to obtain the temperature measurement from the sensor 63 and execute a program comprising program code instructions in order to calculate the pressure of the ammonia stream from the temperature measurement obtained. For this calculation, the control device 4 may use a Clausius-Clapeyron relationship, or any other type of (theoretical or experimental) relationship governing the sorption of the ammonia on the compound 62. The Clausius-Clapeyron relationship may be stored in a memory internal or external to the control device 4. As illustrated in FIG. 2, the control device 4 is connected to one of the ports of the electrical connector 64. The control device 4 reads at this port (and in this sense obtains) the temperature measurement from the sensor 63. The control device 4 is also configured in order to detect a difference between the estimated pressure of the ammonia stream and a pressure setting provided, for example, by the electronic control unit 2. If a difference is detected, the control device 4 adjust the heating power of the heating device 53 in order to compensate for this difference.

In the embodiment illustrated in FIG. 2, the casing 61 is provided with thermal insulation 65 placed inside the casing. The thermal insulation 65 goes right round the casing. The pressure of ammonia present around the compound 62 and the thermal insulation 65 brings the sensor 63 to the equilibrium temperature given by the Clausius-Clapeyron law. Phase-change materials (PCM), the temperatures of which are located around the desired temperature, make it possible to obtain a particular signature during the time analysis of the temperature signal (plateaus at 2 phase-change temperatures if 2 phase-change materials are used. The 2 phase-change materials are referenced 66 and 67 in FIG. 2). For example, if a pressure of 2.8 bar absolute is desired, materials will be taken for which the phase change takes place at temperatures that correspond via the Clausius-Clapeyron law of the salt to 2.5 and 3.1 bar. The presence of the phase-change materials thus makes it possible to accurately estimate the pressure with respect to a predefined pressure range that corresponds to the phase-change temperatures of the PCM materials used.

In one embodiment variant, the pressure measuring device 6 according to invention may comprise several temperature and/or heat flux sensors.

Advantageously, the control device 4 may use the temperature measurement(s) (i.e. instantaneous measurements) and/or a history of temperature measurements in order to diagnose a possible leak in the storage system 5 or to detect a malfunction of a component of the system.

1-15. (canceled)

16. A device for measuring a pressure of a gas in a pollution control or energy storage system comprising:
a casing including an inlet and an outlet;
a compound placed inside the casing and configured to absorb at least one portion of gas entering the casing via the inlet, the portion of the gas not absorbed exiting the casing through the outlet;
at least one sensor configured to measure a temperature of the compound;
a processing unit configured to determine a pressure of the gas by using the temperature measurement of the sensor and a predetermined pressure/temperature relationship.

17. The device as claimed in claim 16, wherein the compound is a solid.

18. The device as claimed in claim 17, wherein the compound is an Mg, Ba or Sr chloride.

19. The device as claimed in claim 16, wherein the pressure/temperature relationship is a Clausius-Clapeyron relationship.

20. The device as claimed in claim 16, wherein the casing is made from a thermoplastic selected from polyamides or polyphthalamides or copolymers thereof.

21. The device as claimed in claim 16, wherein the casing comprises an electrical connector via which the sensor is powered and/or via which the processing unit obtains the temperature measurement from the sensor.

22. The device as claimed in claim 16, wherein the casing comprises means for guiding the gas toward the inlet.

23. The device as claimed in claim 16, wherein the casing comprises thermal insulation means.

24. The device as claimed in claim 16, wherein the casing comprises at least one phase change material.

25. The device as claimed in claim 16, connected to a storage component or gas distribution component included in the pollution control or energy storage system.

26. The device as claimed in claim 25, wherein the storage component or gas distribution component is configured to contain a salt on which the gas is stored by sorption.

27. The device as claimed in claim 16, wherein the gas is ammonia.

28. The device as claimed in claim 16, wherein the gas is hydrogen.

29. A pollution control system comprising at least one pressure measuring device as claimed in claim 16.

30. An energy storage system comprising at least one pressure measuring device as claimed in claim 16.
